(19) United States
(12) Patent Application Publication
(10) Pub. No.: US 2022/0218506 A1
(43) Pub. Date: Jul. 14, 2022

(54) Shoe Brace with a Long Support Rod

(71) Applicant: Americo Andrade, Lincolnton, NC (US)

(72) Inventor: Americo Andrade, Lincolnton, NC (US)

(21) Appl. No.: 17/148,578

(22) Filed: Jan. 14, 2021

(51) Int. Cl. A61F 5/01; A43B 7/00

(52) U.S. Cl. CPC A61F 5/0111; A43B 7/00

(57) ABSTRACT

This disclosure relates to shoe brace with long supportive rod for a user to use it with wearing footwear. The shoe brace includes a shoe brace, a long supportive rod and adjustable straps. The disclosure has a lower leg holder made up of fabric which surrounds the lower leg and ankle portion by buckling the adjustable straps. Further it has long supportive rod extending downwardly towards the footwear which slips in under the shoe lace portion just above the shoe tongue. The shoe brace acts as retaining member secured to the foot strut juxtaposed to the footwear restraining a rearward pivotal movement of the brace. The invention is a shoe brace with a Long Support Rod inserted in between the shoelaces and the tongue of a shoe to support, secure and suspend the shoe in the upwards position.

### BACKGROUND

### Field of the Invention

The present invention relates generally to apparatus for The present invention relates to a Shoe Brace with a Long Support Rod inserted in between the shoelaces and the tongue of a shoe to support, secure and suspend the shoe in the upwards position.

The current design and invention of the device is more pertinent to the concept of shoe braces, protective wear and similar articles worn on the body or appendages of humans. More particularly, these inventions relate generally to a customized fabric based shoe brace closure systems that provide equilibrated tightening pressure for a brace, protective wear with a support rod.

### Discussion of Related Art

Orthopedic supports and braces are typically used to stabilize and protect various limbs of the human anatomy from sustaining or worsening an injury. Typically, orthopedic braces have been used on elbows, wrists, knees and ankles. The purpose of an orthopedic brace is to reduce strain on the injured limb while permitting the limb to continue its function, minimizing the risk of either a more damaging injury or renewing an old injury.

A common remediation technique for foot drop involved the employment of an ankle foot orthosis, or brace. The goal of bracing is to provide patients with a more normal and comfortable gait. These devices often require professional fitting, which may imply taking of impressions of the affected foot, and customized shoes. Significant delays can occur between the fitting and receipt of the customized orthosis.

Several braces have been developed for foot drop. Short leg fixed braces do not fit into the footwear, do not flex at ankle joint, and do not allow plantar flexion nor dorsiflexion, i.e. they do not provide quite as natural of a gait. Energy return braces also fit inside the footwear and use a natural flex built into the material of the brace to provide assist in dorsiflexion but don't help the shoe to be in upright position all time and if they do so, they do not have a soft fabric material. These braces are often made of polymers or carbon graphite materials.

One significant problem with orthopedic leg or shoe braces used during day to day activities is the tendency of the braces to shift as adjacent muscles tense and relax. When this problem is approached by tightening a conventional strapping mechanism, discomfort and restriction of circulation occurs. Even partial shifting of a typical orthopedic shoe brace interferes with its proper operation, and a comfortable flexible shoe brace is required to keep the shoe and foot in upright position.

Another problem with conventional shoe braces is the bulky fittings used to couple straps. D-ring strap fittings usually depend from fittings that are attached to the brace by rivets or other fastening structures. Whenever the cuff structures have such extraneous additional structure for fitting the straps, user discomfort is increased from the added bulk and complexity, and a low profile is difficult to achieve. A straight rod that can fit in the shoes directly was not available. Further if any support was available, did not had the availability in different type of materials embedded with fabric.

In the previous art related to shoe braces, another problem with the conventional shoe braces was the lack of even compression of the soft tissues of the foot against the internal structure of the bones whose position and movement are to be controlled. As force is applied to a conventional brace, the brace tightens selectively near the tightened strap. This is particularly the case with a conventional brace that uses full circumference straps around. On contrary, if the braces were not tightened, the upright position of foot or walking shoes was not achievable. The typical strapping mechanism often does not adequately distribute the tightening force along the length of a tightening zone and keep the foot upright. Therefore, there are areas of higher and lower tension throughout the brace.

Another drawback associated with conventional strapping mechanisms is that it is often difficult to untighten or redistribute tension, as the wearer must loosen and readjust the straps and brace positioning.

Common shoe braces do not accommodate full support of a shoe in the upwards position. This invention relates to a shoe brace that offers support and secures a shoe with good footing and suspends the shoe in the upwards position with its Long Support Rod inserted in between the shoelaces and the tongue of a shoe. The support rod lays down underneath the shoe laces and above the tongue of the shoe. This shoe brace is suitable for outdoor and indoor use and is covered and made of fabric material and a plastic and/or metal and/or aluminum rod insert and adjustable belt straps with buckle loops.

However, to Applicant's knowledge, none of these prior art devices has been found to be completely suitable to meet this need. Hence, the inventor of the present invention purposes to resolve and surmount existent technical difficulties to eliminate the aforementioned shortcomings of prior art.

The previous art and invention in this field have limited or not well designed. With reference to previous inventions or utility of such system associated the current invention this holds its own uniqueness and usability in its domain. With all previous embodiments in the field did not provide such facilitation and hence the idea has its own novelty.

### SUMMARY

In light of the disadvantages of the prior art, the following summary is provided to facilitate an understanding of some of the innovative features unique to the present invention and is not intended to be a full description. A full appreciation of the various aspects of the invention can be gained by taking the entire specifications, claims, drawings, and abstract as a whole.

The primary object of the invention is related to the Shoe Brace with a Long Supportive Rod that is inserted in between the shoelace and the tongue of a shoe. The upper part of the Shoe Brace is wrapped around the ankle for support and the lower part of the Brace is its Long Supportive Rod that is inserted underneath the shoelaces and above the tongue of a shoe.

When shoelaces are tied, and the straps are latched it fully secures the rod of the brace with the shoe. The Shoe Brace with its Long Supportive Rod is covered with fabric material and has adjustable belt straps with buckle loops.

One of the embodiment of the current invention is to provide soft shoe braces that are primarily designed to provide cushioning and protection. It offers padding, shock attenuation, and reduction of friction shear forces. The shoe braces are made up of softer material which is fabric and is an easy gear kit for the foot. The shoe braces are designed to comfort the user with soft orthoses including insensate foot and fixed deformities.

Another embodiment of the invention is the long support rod which is made up of rigid material and is designed to provide excellent arch support and control for flexible deformities, and is durable in nature. The support rod is designed to control or decrease motion and keep the foot in upright position. The main advantage is that it is attachable to the shoe. The long supportive rod can be inserted underneath the shoelaces and above the tongue of a shoe in a manner that it keeps the foot upright and controls the motion of the foot.

This Summary is provided merely for purposes of summarizing some example embodiments, so as to provide a basic understanding of some aspects of the subject matter described herein. Accordingly, it will be appreciated that the above-described features are merely examples and should not be construed to narrow the scope or spirit of the subject matter described herein in any way. Other features, aspects, and advantages of the subject matter described herein will become apparent from the following Detailed Description, Figures, and Claims. In light of the disadvantages of the prior art, the following summary is provided to facilitate an understanding of some of the innovative features unique to the present invention and is not intended to be a full description.

### BRIEF DESCRIPTION OF DRAWING

The invention will be described with respect to the following drawings, in which like reference numerals represent like features throughout the figures. The accompanying figures, where like reference numerals refer to identical or functionally similar elements throughout the separate views, together with the detailed description below, are incorporated in and form part of the specification, and serve to further illustrate embodiments of concepts that include the claimed invention, and explain various principles and advantages of those embodiments.

FIG. 1 is a perspective schematic front view of the described Shoe Brace with a Long Supportive Rod. The figure further elaborates the adjustable belt straps with buckle loops from the front side. The figure shows the shoe brace system in unbuckled position.

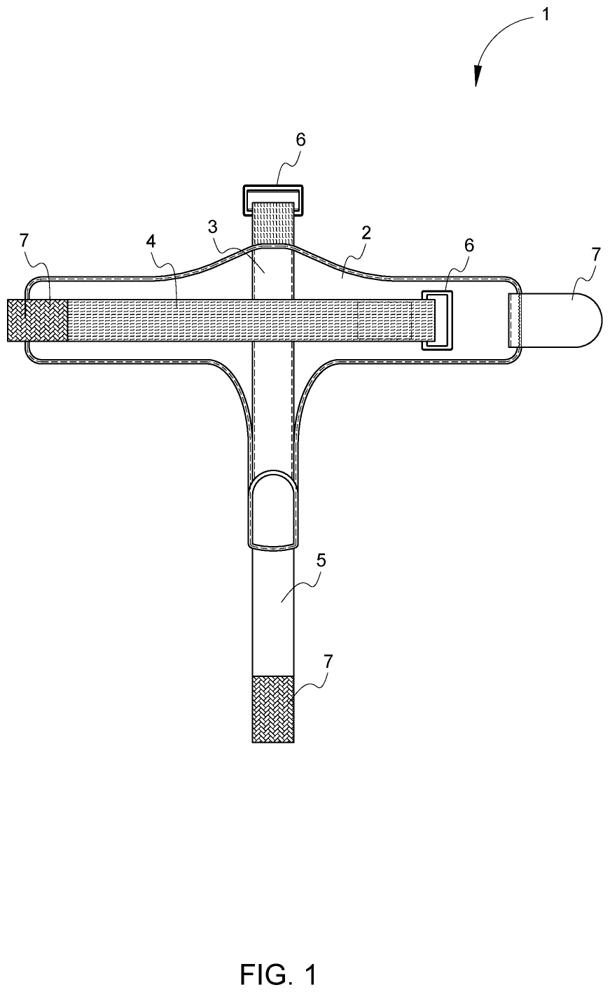

FIG. 2 is a perspective schematic back view of the described Shoe Brace with a Long Supportive Rod. The figure further elaborates the adjustable belt straps with buckle loops from the rear side. The figure shoes the shoe brace system in unbuckled position.

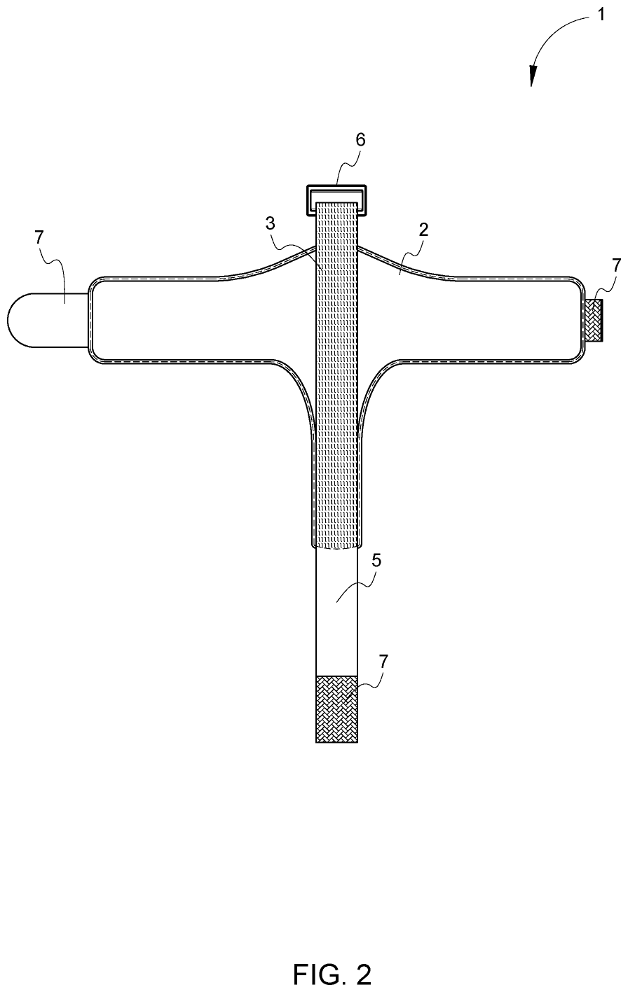

FIG. 3 is a perspective schematic left view and is the same view of the left view of the embodiment of FIG. 1. The figure further elaborates the adjustable belt straps with buckle loops from the left side. The figure shows the shoe brace system in buckled position and the way they would be buckled around the lower foot of the user and the long support rod being in shoe insert position.

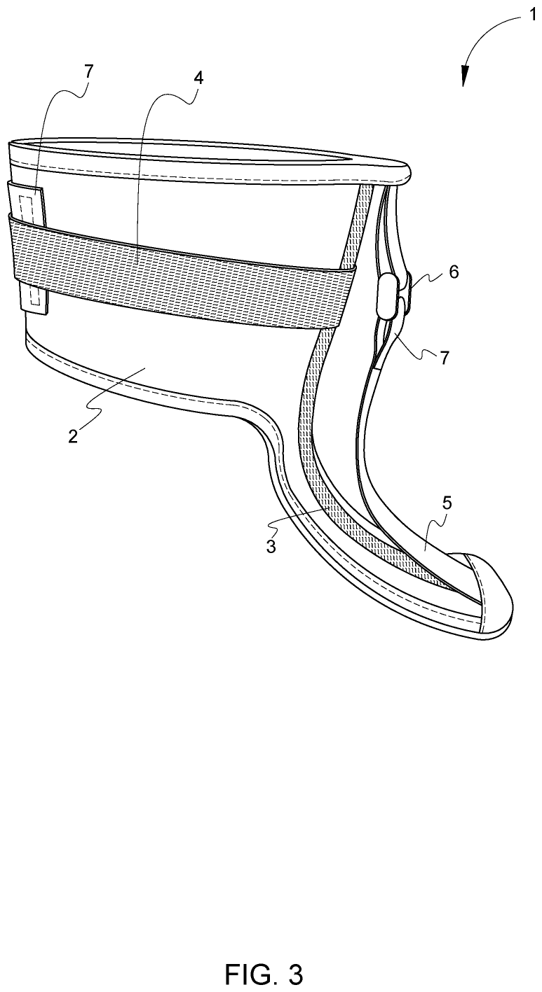

FIG. 4 is a perspective schematic right view and is the same view of the right view of the embodiment of FIG. 1. The figure further elaborates the adjustable belt straps with buckle loops from the right side. The figure shows the shoe brace system in buckled position and the way they would be buckled around the lower foot of the user and the long support rod being in shoe insert position.

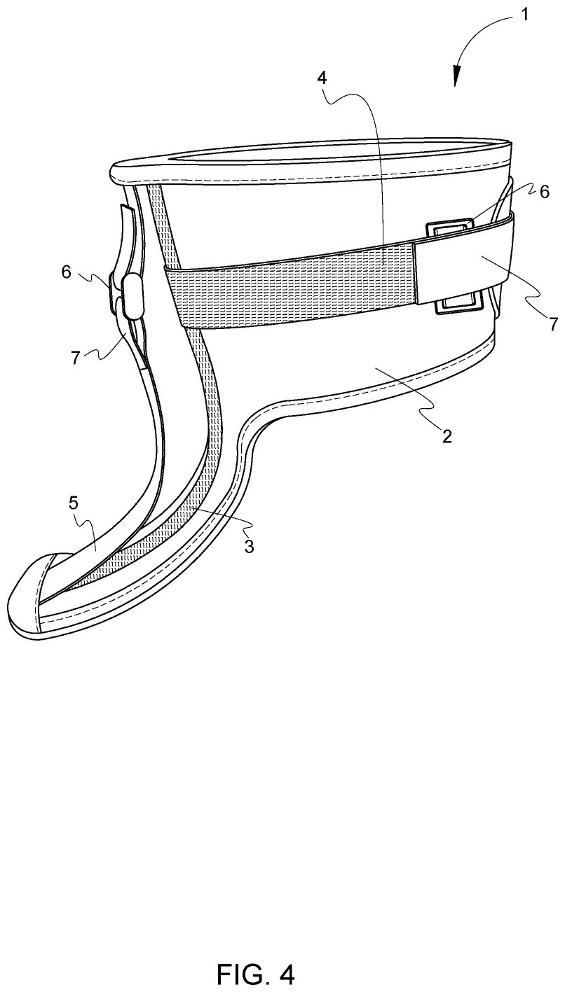

Skilled artisans will appreciate that elements in the figures are illustrated for simplicity and clarity and have not necessarily been drawn to scale. For example, the dimensions of some of the elements in the figures may be exaggerated relative to other elements to improve understanding of embodiments of the present invention. The apparatus and method components have been represented where appropriate by conventional symbols in the drawings, showing only those specific details that are pertinent to understanding the embodiments of the present invention so as not to obscure the disclosure with details that will be readily apparent to those of ordinary skill in the art having the benefit of the description herein.

### DETAILED DESCRIPTION

Detailed descriptions of the preferred embodiment are provided herein. It is to be understood, however, that the present invention may be embodied in various forms. Therefore, specific details disclosed herein are not to be interpreted as limiting, but rather as a basis for the claims and as a representative basis for teaching one skilled in the art to employ the present invention in virtually any appropriately detailed system, structure or manner.

Detailed descriptions of the preferred embodiment are provided herein. It is to be understood, however, that the present invention may be embodied in various forms. Therefore, specific details disclosed herein are not to be interpreted as limiting, but rather as a basis for the claims and as a representative basis for teaching one skilled in the art to employ the present invention in virtually any appropriately detailed system, structure or manner.

The present invention represents the Shoe Brace with a Long Supportive Rod. The Shoe Brace with a Long Supportive Rod that is inserted in between the shoelace and the tongue of a shoe. The upper part of the Shoe Brace is wrapped around the ankle for support and the lower part of the Brace is its Long Supportive Rod that is inserted underneath the shoelaces and above the tongue of a shoe.

According to a general aspect, the invention provides an orthotic foot brace for a person wearing the footwear. The shoe brace with long support rod has two parts. The first part is a lower leg holder securable around a lower leg and ankle portion of the user and is secured to the through its laces and further extends downwardly towards the footwear. The shoe brace acting as a strut has a rear section secured to the vertical side of the fabric based brace. The second part is the long support rod which directly slides under the shoe lace portion and the shoe tongue and holds the foot from unnecessary movement. This portion is made of a rigid material composed of plastic, aluminum, graphite to provide strength and hold the foot and footwear in upward position.

Another embodiment of the current invention is that it has at least one of a median section and a lateral section located on a respective side of the footwear and extending outwardly thereof, and a front section securable to an instep section of the footwear; and at least one brace retaining member secured to the foot strut juxtaposed to the footwear, and having at least a section extending on the median side of the footwear and having at least a section extending on the lateral side of the footwear, outwardly thereof, and restraining a rearward oriented pivotal movement of the brace.

Another embodiment of the invention is to provide shoe brace for a the user wearing a footwear comprising: a frame made up of fabric including a lower leg holder securable to a lower leg and ankle portion of the user. It further provides a vertical section secured to the lower leg holder and extending downwardly towards the footwear. The shoe brace has a foot section secured to the vertical section, extending outwardly of the footwear and forwardly towards an instep section of the footwear and securable to the instep section of the footwear.

The invention has long support rod making a brace that secures the foot section, extending from a median side of the footwear. The rod slides and gets inserted between the shoe laces above the tongue of the shoe. It is present at the front side of the footwear holding the shoe in upward position. The overall shoe brace offers support to the foot and secures a shoe with good footing and suspends the shoe in the upwards position.

This shoe brace is suitable for outdoor and indoor use and holds its utility for household and outdoor usage both. The shoe brace is covered and made of fabric material to provide comfort and firm holding to the lower leg and ankle portion. The supportive rod is made up of rigid material and may be made up of plastic, metal, aluminum material as suitable to the user and subject to upgrade when required. The rod inserts in the footwear and has adjustable belt straps with buckle loops. The adjustable belt straps with buckle loops help the user to adjust the shoe brace according to ease and are connected directly with fabric material for support to the foot from the rear end.

The Long Supportive Rod that is inserted underneath the shoelaces and above the tongue of a shoe will accommodate a better secured application to support the shoe and securely suspend the shoe in the upwards position. When shoelaces are tied, and the straps are latched it fully secures the Rod of the brace with the shoe. The Shoe Brace with its Long Supportive Rod is covered with fabric material and has adjustable belt straps with buckle loops.

The present invention seeks to provide a solution to when a foot is unable to support a shoe on its upward position while walking to prevent tripping. While a specific embodiment has been shown and described, many variations are possible. With time, additional features may be employed. The particular shape or configuration of the platform or the interior figuration may be changed to suit the system or equipment with which it is used.

This detailed description in connection with the drawings is intended principally as a description of the presently preferred embodiments of the invention, and is not intended to represent the only form in which the present invention may be constructed or utilized. The description sets forth the designs, functions, means, and methods of implementing the invention in connection with the illustrated embodiments. It is to be understood, however, that the same or equivalent functions and features may be accomplished by different embodiments that are also intended to be encompassed within the spirit and scope of the invention and that various modifications may be adopted without departing from the invention or scope of the following claims.

Having described the invention in detail, those skilled in the art will appreciate that modifications may be made to the invention without departing from its spirit. Therefore, it is not intended that the scope of the invention be limited to the specific embodiment illustrated and described. Rather, it is intended that the scope of this invention be determined by the appended claims and their equivalents.

The invention claimed is:

1. A shoe brace with a long support rod for use on a user having a lower leg leading to a foot wearing a footwear, the shoe brace comprising:
a lower leg holder attachable around the lower leg of the user made up of fabric material;
a lower leg fabric holder with adjustable straps to secure the lower leg holder and capable of extending downwardly towards the footwear during use;
a long supportive rod secured to the lower leg fabric holder brace, capable of extending therefrom to the front side of the footwear, getting inserted under the shoe lace portion above the shoe tongue connected to the adjustable straps with buckles. A shoe brace attachment securable to an instep section of the footwear, at a location above a foot of the user when the lower leg holder is secured to the lower leg, the lower leg strut extends downwardly towards the footwear.

2. As per claim (1), brace retaining secured to the foot strut and capable of being juxtaposed to the footwear during use.

3. As per claim (1), a long supportive rod which can be inserted in the footwear under the shoe lace above the tongue securing the shoes to be upright position.

4. An orthotic foot brace as claimed in claim (1), wherein the brace is capable of extending from the median side of the footwear to the lateral side of the footwear, during use.

5. An orthotic foot brace as claimed in claim (1), wherein the shoe brace with the long supportive roe is capable of restricting undue rearward pivotal movement.

6. As per claim (1), a shoe brace with fabric and soft material providing extra comfort to the user.

7. A long supportive rod made up of a rigid material to hold the footwear in upward position and restrict undue movement.

8. A shoe brace designed to use with footwear for indoor household and outdoor activities at the same time.

9. A shoe brace as claimed in claim (1) wherein the long support rod is in curved form adjustable and attachable to the footwear.
